(19) United States
(12) Patent Application Publication
(10) Pub. No.: US 2005/0161050 A1
(43) Pub. Date: Jul. 28, 2005

(54) Method of performing soojichim acupuncture using bodycopy

(76) Inventor: Mi-Seon Song, Kyeongki-Do (KR)

Correspondence Address: SEYFARTH SHAW, 55 EAST MONROE STREET, SUITE 4200, CHICAGO, IL, 60603-5803, US

(21) Appl. No.: 10/511,076

(22) PCT Filed: Apr. 10, 2003
(86) PCT No.: PCT/KR03/00719

(30) Foreign Application Priority Data
Apr. 10, 2002 (KR) 10-2002-0019485

(51) Int. Cl. A61B 19/00

(52) U.S. Cl. 128/898

(57) ABSTRACT

Although a preferred embodiment of the present invention has been described for illustrative purposes, those skilled in the art will appreciate that various modifications, additions and substitutions are possible, without departing from the scope and spirit of the invention as disclosed in the accompanying claims.

### TECHNICAL FIELD

The present invention relates in general to a method enabling a person requiring Soojichim acupuncture to perform it by oneself using a copy of the person's body. More particularly, the present invention relates to a method by which a person who has insufficient professional knowledge on acupoint locations and Soojichim acupuncture therapy and also has a difficulty to call on a specialist in Chinese medicine due to the inconvenience of moving can perform Soojichim acupuncture by oneself at home using a copy of the person's body.

### BACKGROUND ART

Unlike general drug therapy, Soojichim acupuncture, which is also called Koryo hand acupuncture or Korean hand acupuncture, has no side effects upon its application and does not cause sequelae or tolerance even upon long-term application. In addition, it is conducted in an easy manner and has excellent effects. For these reasons, it is advantageous in that it can be widely applied to general people.

On the other hand, in order to perform Soojichim acupuncture, there is required thorough knowledge of the acupoint locations effective for a disease on which Soojichim acupuncture can be applied. The acupoint locations significantly vary depending on a disease and are complicated, and thus, Soojichim acupuncture has a disadvantage in that only a person who had learned professional education on Soojichim acupuncture can easily perform Soojichim acupuncture. For this reason, Soojichim acupuncture is performed by only a specialist in Soojichim acupuncture or Chinese herbal medicine in a limited manner without being propagated to general people due to problems of time and cost required for the professional education.

Meanwhile, in attempts to solve these problems, Korean Patent Application No. 1996-71491 discloses an apparatus for guiding Soojichim acupuncture needles to acupoint locations, Korean Patent Application No. 1997-34508 discloses a system for supporting recognition of acupoint locations and a method of performing Soojichim acupuncture using the same, Korean Patent Application No. 2000-27889 discloses a human body diagram, the classification of diseases, and a method of performing and learning Soojichim acupuncture using the information on hand meridians, and Korean Patent Application No. 2000-25576 discloses an apparatus for indicating acupoint locations. However, in these prior arts, the acupoint locations are identified by Internet, or recognized by an expensive system or apparatus, which is used in a complex manner.

Thus, these prior arts have problems in that they are unsuitable for use in an old and feeble person among general people requiring Soojichim acupuncture or in an area having a difficulty to use Internet, and also uneconomical.

### DISCLOSURE OF THE INVENTION

Accordingly, the present invention has been made to solve the above-mentioned problems occurring in the prior art, and an object of the present invention is to provide a method enabling a person requiring Soojichim acupuncture for health maintenance and treatment to easily perform it by oneself at home by forming a copy of the person's body, and marking acupoint locations on the body copy by force of a specialist in Chinese medicine, and then performing Soojichim acupuncture according to the marked acupoint locations.

To achieve the above object, the present invention provides a method enabling a person requiring Soojichim acupuncture to perform it by oneself, which comprises the steps of: forming a copy of the body of the person requiring Soojichim acupuncture; marking acupoint locations on the body copy by force of a specialist in Chinese medicine; attaching the body copy to the corresponding portion of the body; and performing Soojichim acupuncture according to the marked acupoint locations by the person requiring Soojichim acupuncture.

### BRIEF DESCRIPTION OF THE DRAWINGS

A preferred embodiment of the present invention will now be described in detail with reference to the accompanying drawings, in which:

FIG. 1 is a flowchart illustrating a process of forming a copy of the human body according to the present invention;

FIGS. 2a and 2b schematically show a process of forming a copy of the human body according to the present invention; and

FIG. 3 shows a copy of the human body according to the present invention, on which acupoint locations were marked.

### BEST MODE FOR CARRYING OUT THE INVENTION

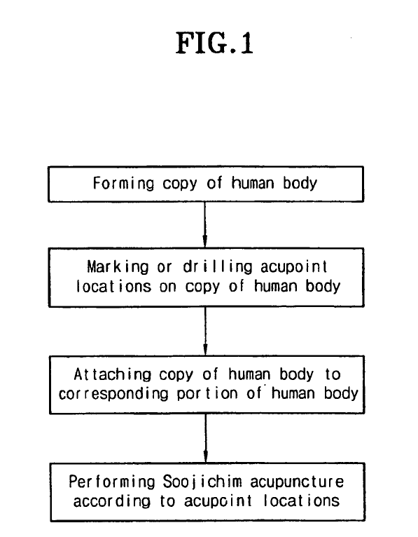

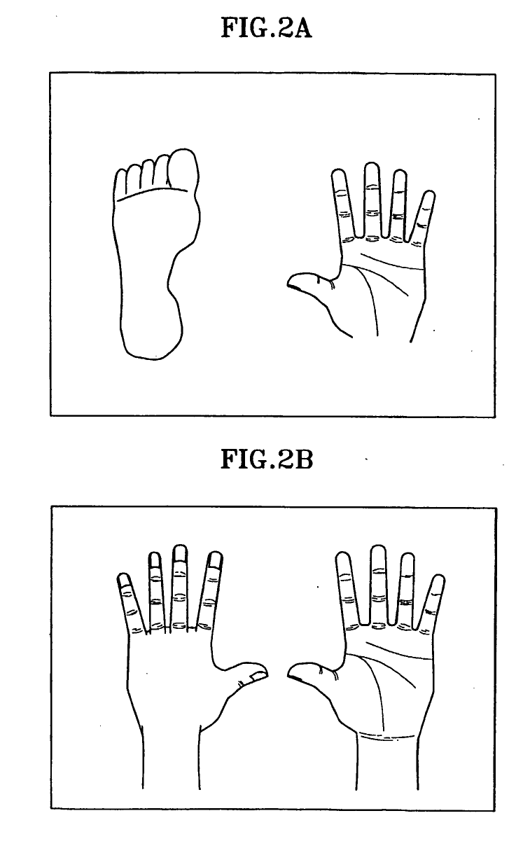

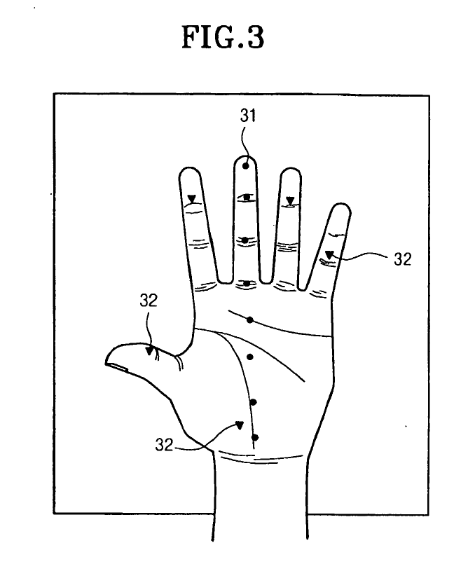

FIG. 1 is a flowchart illustrating a process of forming a copy of the human body according to the present invention; FIGS. 2a and 2b schematically show a process of forming a copy of the human body according to the present invention; and FIG. 3 shows a copy of the human body according to the present invention, on which acupoint locations were marked.

First, as shown in FIG. 2a, an instantaneous molding is made. Next, as shown in FIG. 2b, a copy of the human body is formed using the instantaneous molding.

In this case, the body copy is formed in the same manner as a body copy that is mainly used in body copy shops or medical offices, which are currently managed in various areas. In other words, as shown in FIG. 2a, the instantaneous molding is made by a conventional method allowing precious expression of fingerprints or pores of any desired body portions, such as hand, foot, ear and the like, and then, as shown in FIG. 2b, the body copy is formed using the instantaneous molding.

The instantaneous molding of the body is easily made at home by means of an instantaneous molding kit. Alternatively, it is made in the body copy shop in an inexpensive, easy and rapid manner. Generally, the instantaneous molding is made from vegetable silicon, synthetic clay, synthetic resin or the like.

Such an instantaneous molding can precisely express the body portions, and also if once it is made, it is not deformed so that it can be stored for a long period of time. Thus, after the body copy which was formed on the basis of the instantaneous molding is used in Soojichim acupuncture, the used body copy is re-used in subsequent Soojichim acupuncture. If the body copy gets damaged, there can be used a new body copy which is formed using the stored instantaneous molding.

The body copy of hand, foot, ear or the like formed as described above is sent to a specialist in Chinese medicine or Soojichim acupuncture. By force of the specialist, acupoint locations effective for disease treatment are marked or drilled on the body copy in view of the age or physical conditions of a person requiring Soojichim acupoint.

In addition to the acupoint locations, as shown in FIG. 3, a reference point is marked on the body copy in order to prevent the marked acupoint locations of the body copy from being deviated from the corresponding acupoint locations of the body upon attachment of the body copy to the body.

During safekeeping of the body copy in the home, if health maintenance or treatment recommended by a medical specialist is required, Soojichim acupuncture is performed on the exact locations of acupoints using the body copy.

As a result, a person requiring Soojichim acupuncture can perform Soojichim acupuncture on the exact locations of acupoints by oneself using the body copy without calling on a specialist in Chinese medicine or Soojichim acupuncture. In particular, an old and feeble person or other patients having the difficulty of moving can receive Soojichim acupuncture from a family without calling on a dispensary of Chinese medicine.

In addition to Soojichim acupuncture, moxibustion or press pellets, etc., may also be applied to the acupoint location using the body copy.

### INDUSTRIAL APPLICABILITY

As apparent from the foregoing, the present invention is advantageous in that it enables a person having no professional knowledge on the acupoint locations to perform Soojichim acupuncture in a simple and easy manner, and particularly, it enables a person having the difficulty of moving to perform Soojichim acupuncture on the exact locations of acupoints at home without calling on a specialist in Soojichim acupuncture.

In addition, the present invention is advantageous in that it does not require a special apparatus or system to recognize the acupoint locations, and also the produced instantaneous molding can be stored for a long period of time, so that the present invention is highly economical.

1. A method enabling a person requiring Soojichim acupuncture to perform it by oneself using a copy of the person's body, which comprises the steps of:
forming a copy of the body of the person requiring Soojichim acupuncture;
marking acupoint locations on the body copy by force of a specialist in Chinese medicine;
attaching the body copy to the corresponding portion of the body; and
performing Soojichim acupuncture according to the marked acupoint locations by the person requiring Soojichim acupuncture.

2. The method of claim 1, which further comprises the steps of marking a reference point for attachment, on the body copy, in the step of marking the acupoint locations.
